(19) United States
(12) Patent Application Publication
(10) Pub. No.: US 2018/0021183 A1
(43) Pub. Date: Jan. 25, 2018

(54) DIAPER SENSOR, MANUFACTURING METHOD THEREOF AND DIAPER

(71) Applicant: SHENZHEN HYAN MICROELECTRONICS CO., LTD., Shenzhen, Guangdong (CN)

(72) Inventor: Yujie TENG, Shenzhen, Guangdong (CN)

(21) Appl. No.: 15/320,660

(22) PCT Filed: Mar. 18, 2016
(86) PCT No.: PCT/CN2016/000146

§ 371 Date: Dec. 20, 2016

(30) Foreign Application Priority Data
Apr. 17, 2015 (CN) 201510185155.1

(51) Int. Cl. A61F 13/42; A61F 13/49; A61F 13/47

(52) U.S. Cl. CPC A61F 13/42; A61F 13/47; A61F 13/49

(57) ABSTRACT

The invention relates to a diaper sensor and a manufacture method therefor. The sensor comprises a detection electrode and a hydrophilic first substrate. The detection electrode comprises a first metal strip belt which is arranged on a first surface of the first substrate and in the shape of a straight line, and a second metal strip belt and a third metal strip belt which are located on the two sides of the first metal strip belt and are parallel to the first metal strip belt. The first, second, and third metal strip belts are correspondingly provided with first, second and third conductive connecting parts. The middle portion of the second metal strip belt or the third metal strip belt is provided with a first through hole dividing the metal strip belt into two sections.

### FIELD OF THE INVENTION

The present invention relates to an intelligent diaper, in particular to a diaper sensor and a manufacturing method therefor, and a diaper adopting said sensor.

### BACKGROUND OF THE INVENTION

The diaper disclosed by the present invention generally refers to various paper diapers and panty-shape diapers. The paper diapers and panty-shape diapers are increasingly used as daily necessities frequently used by infants and the aged due to the characteristic of convenience in use. However, it is unavailable for a traditional diaper to judge whether or not a wearer has urinated or defecated from the appearance, a caregiver needs to observe the diaper frequently, and therefore, the wearer will often feel uncomfortable because the diaper is not changed timely. By arranging a sensor on the diaper, the caregiver may be reminded by an alarm signal sent after the wearer urinates or defecates, and therefore, said problem can be overcome.

According to a diaper sensor, two detection electrodes are arranged on a hydrophilic substrate, and after urination or defecation, the hydrophilic substrate gets wet, such that the electrical characteristics between the two detection electrodes change, thereby achieving the purpose of detecting whether a wearer urinates or defecates. Generally, the detection electrodes are arranged on the substrate by adopting a printing process or a knitting process, for example: the detection electrodes are formed by printing conductive silver or conductive carbon on the substrate, or knitting metal wires on a cloth substrate. These processing technologies are complicated and relatively high in cost. Furthermore, an existing diaper sensor can be only used for detecting whether a wearer urinates or defecates, and fails to distinguish excrement from urine.

### SUMMARY OF THE INVENTION

An objective of the present invention is to provide a diaper sensor and a manufacturing method therefor, to solve the above-mentioned technical defects of the existing diaper sensor.

The present invention further provides a diaper adopting the diaper sensor.

The specific technical solution of the present invention is as follows:

a diaper sensor comprises a detection electrode and a hydrophilic first substrate, wherein the detection electrode comprises:

a first metal strip belt which is arranged on a first surface of the first substrate and in the shape of a straight line;

a second metal strip belt which is arranged on the first surface of the first substrate, located on a first side of the first metal strip belt, and spaced from and parallel to the first metal strip belt; and

a third metal strip belt which is arranged on the first surface of the first substrate, located on a second side of the first metal strip belt, and spaced from and parallel to the first metal strip belt.

The first, second and third metal strip belts are correspondingly provided with first, second and third conductive connecting parts. The middle portion of the second metal strip belt or the third metal strip belt is provided with a first through hole dividing the metal strip belt into two sections, so as to correspond to three states after defecation, after urination and before defecation and urination of a wearer. Different electrical characteristics are present among the conductive connecting parts.

In the diaper sensor, preferably, the first, second and third conductive connecting parts are close to one end part of the first substrate. The first metal strip belt between the end part and the first conductive connecting part is provided with a second through hole dividing the first metal strip belt into two sections.

In the diaper sensor, preferably, the diaper sensor further comprises a second substrate arranged on the first surface of the first substrate. The first, second and third metal strip belts are clamped between the first substrate and the second substrate.

In the diaper sensor, preferably, the first substrate and/or the second substrate are/is a cloth substrate.

In the diaper sensor, preferably, the first, second and third metal strip belts are aluminum foil strip belts.

A manufacturing method for the diaper sensor comprises the following steps:

arranging a metal foil on the first surface of the first substrate;

removing a part of the metal foil by using a hob die cutting process to form the three metal strip belts, which are spaced from and parallel to each other, on the first surface of the first substrate; and

punching a hole in the middle portion of one metal strip belt on the edge to divide the metal strip belt into two sections.

Preferably, the manufacturing method for the diaper sensor further comprises a step of punching a hole in a position, close to one end, of the middle metal strip belt to divide the metal strip belt into two sections.

In the manufacturing method for the diaper sensor, preferably, the step of arranging the metal foil on the first surface of the first substrate comprises: adhering two ends of the metal foil and fixing the same to the first surface of the first substrate.

A diaper provided by the present invention comprises a diaper body and further comprises the diaper sensor according to any one of claims. The sensor is fixed inside or on the surface of the diaper body, and:

the first, second and third metal strip belts can pass through the crotch of the wearer; and

the first through hole is located between the anus and the genitals of the wearer.

Preferably, the diaper further comprises a communication triggering device, said communication triggering device comprising:

an acquisition unit configured to acquire electrical characteristics among the first, second and third conductive connecting parts;

a processing unit configured to judge whether the wearer urinates or defecates and distinguish excrement from urine according to the acquired electrical characteristics;

a signal generating unit configured to periodically generate set signals; and

a medium- and short-distance wireless communication unit configured to send a result generated by the processing unit and the signals generated by the signal generating unit to a monitoring terminal.

The present invention has the following advantages:

1. not only whether the wearer urinates or defecates can be found timely, but also excrement can be distinguished from urine;

2. as an independent device, the sensor can be conveniently used with an existing paper diaper or panty-shape diaper via an assembling manner, such as adhering or inlaying;

3. the detection electrode is formed on the substrate by adopting a hob die cutting process, and is simple in process and low in cost; and

4. the medium- and short-distance wireless communication unit can realize the communication between the communication triggering device and the monitoring terminal without communication fee, and can monitor whether the wearer goes beyond a monitoring region after being combined with the signal generating unit.

### BRIEF DESCRIPTION OF THE DRAWINGS

FIG. 1 is a structural schematic drawing of some embodiments of the diaper sensor; and

FIG. 2 is an A-A sectional view of the diaper sensor.

### DETAILED DESCRIPTION OF THE PREFERRED EMBODIMENTS

The present invention will be further illustrated below in conjunction with the drawings and embodiments.

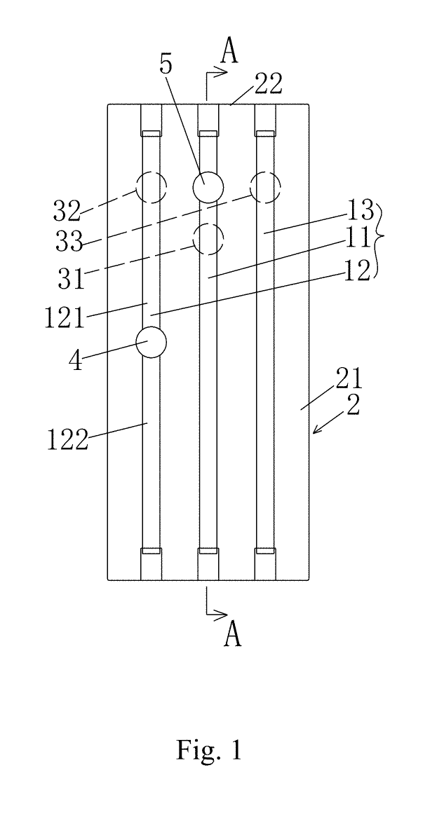

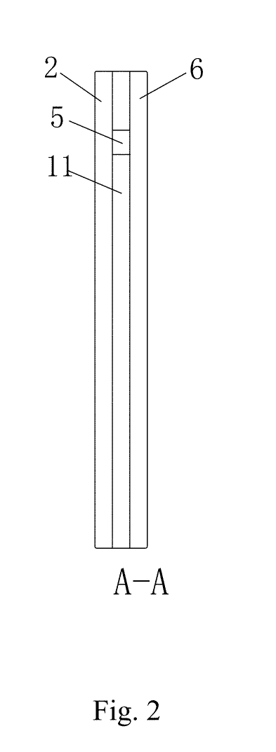

FIGS. 1-2 schematically represent the structure of some embodiments of the diaper sensor, wherein no second substrate is illustrated in FIG. 1 so as to show the internal structure clearly. As shown in FIGS. 1-2, the diaper sensor comprises a detection electrode 1 and a hydrophilic first substrate 2.

The detection electrode 1 comprises a first metal strip belt 11, a second metal strip belt 12 and a third metal strip belt 13. The first metal strip belt 11 is arranged on a first surface 21 of the first substrate 2 and in the shape of a straight line. The second metal strip belt 12 is arranged on the first surface 21 of the first substrate 2, and is located on a first side of the first metal strip belt 11, and spaced from the first metal strip belt 11 (that is, the two metal strip belts are spaced by a certain distance) and parallel to the first metal strip belt 11. The third metal strip belt 13 is arranged on the first surface 21 of the first substrate 2, and located on a second side of the first metal strip belt 11, and spaced from and parallel to the first metal strip belt 11.

The first, second and third metal strip belts 11, 12, 13 are correspondingly provided with first, second and third conductive connecting parts 31, 32, 33. The middle portion of the second metal strip belt 12 is provided with a first through hole 4 dividing the second metal strip belt 12 into a first section 121 and a second section 122. Because only the first section 121 is electrically connected to the second conductive connecting part 32, only the first section 121 is an effective part of the second metal strip belt 12, and actually the second section 121 is an ineffective part.

When the diaper is used, the first, second and third metal strip belts 11, 12, 13 pass through the crotch of the wearer, and the first through hole 4 is located between the anus and the genitals of the wearer. Therefore, a portion over the first through hole 4 (hereinafter referred to as a substrate upper portion) is arranged at the genitals of the wearer, and a portion below the first through hole 4 (hereinafter referred to as a substrate lower portion) is arranged at the anus of the wearer. As such, after the wearer urinates, the substrate upper portion gets wet, such that the resistance among the first, second and third metal strip belts decreases (relative to the case where the wearer does not urinate or defecate); and after the wearer defecates, the substrate lower portion changes, and due to the blocking effect of the first through hole 4, only the resistance between the first and third metal strip belts 11, 13 decreases (relative to the case where the wearer does not urinate or defecate) at this moment, and the resistance between the second metal strip belt 12 (referring to the effective portion) and other metal strip belt does not change (relative to the case where the wearer does not urinate or defecate). Therefore, by identifying the electrical characteristics (resistance or current) among the three conductive connecting parts 31, 32, 33, whether the wearer urinates or defecates can be learnt and excrement can be distinguished from urine.

It can be understood that said functions may also be realized by forming the first through hole 4 in the middle portion of the third metal strip belt 13.

Referring to FIG. 1, in this embodiment, the first, second and third conductive connecting parts 31, 32, 33 are close to one end part 22 (the upper end) of the first substrate 2. To avoid short-circuiting during electric connection between the sensor and other device, the first metal strip belt between the end part 22 and the first conductive connecting part 31 is provided with a second through hole 5 dividing the first metal strip belt 11 into two sections.

Further, the diaper sensor further comprises a second substrate 6 arranged on the first surface of the first substrate 2. The first, second and third metal strip belts 11, 12, 13 are clamped between the first substrate 2 and the second substrate 6.

The first substrate 2 is a cloth substrate preferably, and may also be other hydrophilic substrates, such as a paper substrate. The second substrate 6 is a cloth substrate preferably, and may also be other substrates, such as a paper substrate.

The first, second and third metal strip belts 11, 12, 13 are aluminum foil metal strip belts preferably.

The sensor may be formed using a hob die cutting process, rather than other forming process, such as printing or knitting, which is complicated and high in cost, because the three metal strip belts constituting the detection electrode are parallel to each other. The manufacturing process for the diaper sensor will be further illustrated as below.

A manufacturing method for the diaper sensor comprises the following steps:

arranging a metal foil on the first surface of the first substrate;

removing a part of the metal foil by using a hob die cutting process to form three metal strip belts (namely the first, second and third metal strip belts), which are spaced from and parallel to each other, on the first surface of the first substrate; and

punching a hole in the middle portion of one metal strip belt on the edge (namely the second metal strip belt or the third metal strip belt) so as to form the first through hole dividing the metal strip belt into two sections.

In some embodiments, the manufacturing method further comprises a step of punching a hole in a position, close to one end, of the middle metal strip belt to form the second through hole dividing the metal strip belt into two sections.

Preferably, a metal foil is arranged on the first surface of the first substrate via an adhering manner. For example, the metal foil is arranged on the first surface of the first substrate, and two end parts of the metal foil are fixed with adhesive tapes.

The sensor may be conveniently used with an existing paper diaper and panty-shape diaper via an assembling method, such as adhering or inlaying, to constitute an intelligent diaper.

Specifically, a diaper comprises a diaper body and the diaper sensor. The sensor is fixed inside or on the surface of the diaper body, and: the first, second and third metal strip belts can pass through the crotch of the wearer; and the first through hole is located between the anus and the genitals of the wearer.

In some embodiments, the diaper further comprises a communication triggering device which is detachably arranged on the diaper. The communication triggering device comprises an acquisition unit configured to acquire electrical characteristics among the first, second and third conductive connecting parts; a processing unit configured to judge whether the wearer urinates or defecates and distinguish excrement from urine according to the acquired electrical characteristics; a signal generating unit configured to periodically generate set signals; and a medium- and short-distance wireless communication unit configured to send a result generated by the processing unit and the signals generated by the signal generating unit to a monitoring terminal. The medium- and short-distance wireless communication unit described herein may be a Bluetooth module, or a WIFI module, or an NFC (Near Field Communication) module, or an RFID (Radio Frequency Identification) module, and may also be two or more of them. The monitoring terminal described herein may be a smartphone, a tablet computer or the like. The communication triggering device may be used repeatedly, and the sensor is disposable.

The signal generating unit is combined with the medium- and short-distance wireless communication unit, such that an alarm signal may be generated timely by periodically sending set signals to the monitoring terminal, when the wearer goes beyond a monitoring region. Specifically, when the wearer moves out of a coverage range of the medium- and short-distance wireless communication unit, the monitoring terminal fails to receive the set signals, thereby generating an alarm signal to remind a caregiver that the diaper wearer goes beyond the monitoring region.

A more detailed description is made for the present invention as above in conjunction with the specific embodiments, but these specific descriptions will not be interpreted to limit the protection scope of the present invention. Those skilled in the art may also make other modifications or equivalent replacements according to the description of the present invention. The protection scope of the present invention is subject to the claims.

What is claimed is:

1. A diaper sensor, comprising a detection electrode (1) and a hydrophilic first substrate (2), wherein the detection electrode comprises:
a first metal strip belt (11) which is arranged on a first surface (21) of the first substrate and in the shape of a straight line;
a second metal strip belt (12) which is arranged on the first surface (21) of the first substrate, located on a first side of the first metal strip belt, and spaced from and parallel to the first metal strip belt; and
a third metal strip belt (13) which is arranged on the first surface (21) of the first substrate, located on a second side of the first metal strip belt, and spaced from and parallel to the first metal strip belt;
the first, second and third metal strip belts are correspondingly provided with first, second and third conductive connecting parts (31, 32, 33); the middle portion of the second metal strip belt or the third metal strip belt is provided with a first through hole (4) dividing the metal strip belt into two sections (121, 122), so as to correspond to three states after defecation, after urination and before defecation and urination of a wearer; and different electrical characteristics are present among the conductive connecting parts.

2. The diaper sensor according to claim 1, wherein the first, second and third conductive connecting parts (31, 32, 33) are close to one end part (22) of the first substrate (2). The first metal strip belt between the end part (22) and the first conductive connecting part (31) is provided with a second through hole (5) dividing the first metal strip belt into two sections.

3. The diaper sensor according to claim 1, wherein the diaper sensor further comprises a second substrate (6) arranged on the first surface of the first substrate, and the first, second and third metal strip belts are clamped between the first substrate and the second substrate.

4. The diaper sensor according to claim 3, wherein the first substrate and/or the second substrate are/is a cloth substrate.

5. The diaper sensor according to claim 1, wherein the first, second and third metal strip belts (11, 12, 13) are aluminum foil strip belts.

6. A manufacturing method for the diaper sensor, comprising the following steps:
arranging a metal foil on the first surface of the first substrate;
removing a part of the metal foil by using a hob die cutting process to form the three metal strip belts, which are spaced from and parallel to each other, on the first surface of the first substrate; and
punching a hole in the middle portion of one metal strip belt on the edge to divide the metal strip belt into two sections.

7. The manufacturing method for the diaper sensor according to claim 6, further comprising a step of punching a hole in a position, close to one end, of the middle metal strip belt to divide the metal strip belt into two sections.

8. The manufacturing method for the diaper sensor according to claim 6, wherein the step of arranging the metal foil on the first surface of the first substrate comprises: adhering two ends of the metal foil and fixing the same to the surface of the first substrate.

9. A diaper, comprising a diaper body and further comprising the diaper sensor according to claim 1, wherein the sensor is fixed inside or on the surface of the diaper body, and:
the first, second and second metal strip belts can pass through the crotch of the wearer; and
the first through hole is located between the anus and the genitals of the wearer.

10. The diaper according to claim 9, further comprising a communication triggering device, said communication triggering device comprising:
an acquisition unit configured to acquire electrical characteristics among the first, second and third conductive connecting parts;
a processing unit configured to judge whether the wearer urinates or defecates and distinguish excrement from urine according to the acquired electrical characteristics;
a signal generating unit configured to periodically generate set signals; and
a medium- and short-distance wireless communication unit configured to send a result generated by the processing unit and the signals generated by the signal generating unit to a monitoring terminal.
